(19) United States
(12) Patent Application Publication
(10) Pub. No.: US 2020/0367987 A1
(43) Pub. Date: Nov. 26, 2020

(54) Container Holder For A Dialysis Apparatus

(71) Applicant: FRESENIUS MEDICAL CARE DEUTSCHLAND GMBH, Bad Homburg (DE)

(72) Inventors: Gesa GERCKE, Schweinfurt (DE); Jochen RUECKERT, Roethlein (DE); Thomas FAULHABER, Bergrheinfeld (DE)

(73) Assignee: Fresenius Medical Care Deutschland GmbH, Bad Homburg (DE)

(21) Appl. No.: 16/634,241

(22) PCT Filed: Jul. 23, 2018
(86) PCT No.: PCT/EP2018/069900

§ 371 Date: Jan. 27, 2020

(30) Foreign Application Priority Data
Jul. 27, 2017 (DE) 10 2017 117 070.7

(51) Int. Cl. A61B 50/33; A61M 1/14

(52) U.S. Cl. CPC A61B 50/33; A61M 1/14; A61B 2050/3008; A61M 2209/082; A61M 2209/08

(57) ABSTRACT

The present disclosure relates to a container holder for a dialysis apparatus having at least a first container receptacle, a second container receptacle, and a holding section for movably connecting the container holder to the dialysis apparatus. The first receptacle container is configured to position a first container and the second receptacle container is configured to position a second container.

### CROSS-REFERENCE TO RELATED APPLICATIONS

The present application is the national stage entry of International Patent Application No. PCT/EP2018/069900, filed on Jul. 23, 2018, and claims priority to Application No. DE 102017117070.7, filed in the Federal Republic of Germany on Jul. 27, 2017, the disclosures of which are expressly incorporated herein in its entirety by reference thereto.

### TECHNICAL FIELD

The present disclosure relates to a container holder for use with dialysis apparatuses.

### BACKGROUND

Apparatuses for therapeutic apheresis, liver support therapy, oxygenation, peritoneal dialysis, or hemofiltration or hemodialysis are known from practice. For the potential treatment of the patient, they may be connected to containers which contain liquids used in the treatment.

### SUMMARY

A container holder for a dialysis apparatus encompasses a first container receptacle, which is embodied to position a first container, as well as a second container receptacle for positioning a second container. Optionally, the container holder comprises only the first container receptacle or only the second container receptacle, but no further container receptacles.

In the following, the apparatuses of the aforementioned types will be collectively referred to as dialysis apparatuses.

The container holder comprises a holding section for movably holding the container holder on or at the dialysis apparatus.

The movable holding may optionally be provided as, or ensured by, a folding device, a shifting device, and/or a releasable holding apparatus. A movable holding may therefore be understood in some embodiments also as a releasable holding, in other embodiments it is not releasable.

A movable holding may optionally allow a relative movement between the container holder and the dialysis apparatus.

A releasable holding may optionally be designed such that the container holder may be removable from the dialysis apparatus without using any tools. Preferably, it may be also again arranged to the dialysis apparatus manually, without any tools.

The present disclosure further relates to a dialysis apparatus which comprises a container holder.

The dialysis apparatus encompasses a mounting device which is designed to be movably connected to a holding section of the container holder at a retaining section of the dialysis apparatus.

The apparatus (synonym: a corresponding mechanism, both terms are interchangeable) for folding, shifting or releasing may thereby be provided through corresponding apparatuses at either the dialysis apparatus or at the container holder or at both apparatuses, namely at the dialysis apparatus and at the container holder.

The connection between a main body of the dialysis apparatus and the container holder may be provided on the rear side of the main body, wherein the front side comprises a plurality (i.e., at least two) of operating elements for a treatment. At the front side, there may be arranged e.g., a blood pump, a clamp for a tube which, in use, is blood conveying, and/or holders for a tube which, in use, is blood conveying, and/or for a cassette which, in use, is blood conveying.

In all of the remarks made herein, the use of the expression “may be” or “may have” and so on, is to be understood synonymously with “preferably is” or “preferably has,” and so on respectively, and is intended to illustrate embodiments.

Embodiments may comprise one or several of the features mentioned above and in the following. In this, the features mentioned herein may be, in any combination, the subject of embodiments, unless the person skilled in the art recognizes a specific combination as technically impossible.

Furthermore, embodiments are the subject of the dependent claims.

Whenever numerical words are mentioned herein, the person skilled in the art shall recognize or understand them as indications of numerical lower limits. Unless it leads the person skilled in the art to an evident contradiction, the person skilled in the art shall comprehend the specification for example of “one” as encompassing “at least one”. This understanding is also equally encompassed as the interpretation that a numeric word, for example, “one” may alternatively mean “exactly one”, wherever this is evidently technically possible for the person skilled in the art. Both are encompassed and apply herein to all used numerical words.

Whenever an embodiment is mentioned herein, it is then an exemplary embodiment.

“Top” and “bottom” are indications which, in case of doubt by the person skilled in the art, are to be understood as absolute or relative spatial indications which relate to the orientation of the corresponding component during its intended use.

In some embodiments, the container holder is designed as a tray, e.g., as a tray with exactly one indentation with regard to its surface.

In some embodiments, the container holder is wider and/or deeper than it is high.

In some embodiments, the container holder comprises at least one partition wall between the first container receptacle and the second container receptacle. The partition wall may have the shape of a wall. It may optionally have a different shape than a wall. The partition wall may thus optionally be a separating section. The partition wall is optionally provided.

The partition wall, or sections thereof, may run or extend straight, with one curvature radius or a plurality thereof (e.g., in a cross-section view of the partition wall).

In some embodiments, a partition wall is formed when the first container receptacle and the second container receptacle are arranged on different levels (for example, relative to the base surface of the dialysis apparatus during use). The wall formed in that way, limiting the lower-arranged container receptacle as a side wall at least in section, may be understood as a partition wall in the sense of this description.

In some embodiments, a partition wall is formed by a wall, a bulge or another protrusion extending between the first container receptacle and the second container receptacle.

Due to the partition wall which is arranged to “separate”, liquid that escapes from a container, arranged in the first container receptacle, may be diverted away from the second container receptacle or prevented from entering it, or vice versa.

The container receptacle may comprise exactly two or at least two such partition walls for each the first and the second container receptacle. The partition wall (s) may have an annular form. They may have a closed cross-section (e.g., in a ring-form) or an open cross-section (e.g., in a U-form).

The partition wall, or sections thereof, may be curved. A curved partition wall may be understood as a partition wall with an inconstant height. The curved form may advantageously contribute to saving material.

In some embodiments, the first container receptacle comprises, or is, a drip pan. Such a drip pan serves to collect liquid which has escaped from the first container or originates therefrom, when the container is placed in the first container receptacle.

In some embodiments, the second container receptacle comprises, or is a drip pan. Such a drip pan serves to collect liquid which has escaped from the second container or originates therefrom, when this container is placed in the second container receptacle.

In some embodiments, the container holder encompasses a first base or support surface of the container receptacle and a second base or support surface of the second container receptacle. The first support surface, or sections thereof, which serves for placing the first container, is arranged during use to be lower than the second support surface, or sections thereof, which serves for placing the second container.

In some embodiments, the support surface, or sections thereof, of the first container receptacle and/or the support surface, or sections thereof, of the second container receptacle are arranged during use of the container holder lower than a connecting section, which extends between the first and/or the second container receptacle and connects both container receptacles.

In some embodiments, the first container receptacle of the container holder comprises (during use) an upper edge. This upper edge, or sections thereof, is arranged higher than the support surface, or sections thereof, of the second container receptacle which serves for placing the second container.

The containers—exemplarily used for disinfecting the dialysis apparatus—are placed with their bottom area onto the support surface or in the drip pan of the first and of the second container receptacle.

In this, the containers used during dialysis may comprise rectangular or annular bottom surfaces (or cross-sections, applies also in the following) (alternatively: support surfaces). The basic shape of the support surface and/or of the drip pans may be adapted to these forms.

In particular, the container holder may comprise a rectangular first support surface and/or drip pan for a container having a rectangular bottom area and an annular second support surface and/or drip pan for a container having an annular bottom area.

Alternatively, the container holder may comprise a rectangular first support surface and/or drip pan for a container having a rectangular bottom area and a rectangular second support surface and/or drip pan for a container having a rectangular bottom area.

Further alternatively, the container holder may comprise an annular first support surface and/or drip pan for a container having an annular bottom area and an annular second support surface and/or drip pan for a container having an annular bottom area, or vice versa.

In some embodiments, the holding section encompasses one or several openings. The openings may serve to receive pins, push buttons or securing pins.

In some embodiments, the openings of the holding sections encompass at least one annular opening and/or at least one key-hole shaped or tapered opening.

In some embodiments, the first container receptacle and/or the second container receptacle comprises a free opening or a free interior having an annular cross-section for positioning a container.

In some embodiments, the dialysis apparatus encompasses a mounting device, which comprises at least one pin or one securing pin.

In some embodiments, the mounting device of the dialysis apparatus comprises at least one push button.

In some embodiments, the dialysis apparatus is an ultrafiltration apparatus, hemofiltration apparatus, hemodialysis apparatus and/or hemodiafiltration apparatus.

The dialysis apparatus may optionally comprise, or be connected to, one or several of the following components: arterial line section, venous line section, arterial connection needle, venous connection needle, arterial patient tube clamp, venous patient tube clamp, blood pump, blood filter, arterial pressure sensor, venous drip chamber, venous pressure sensor, substituate pump, substituate liquid reservoir, substituate liquid inlet line, pre-dilution addition site, post-dilution addition site, signal processing unit, control line for the blood pump, and/or control line for the substituate pump.

In some embodiments, the container holder comprises no movable parts.

In some embodiments, the container holder is one piece.

In some embodiments, the container holder is integrally manufactured, for example by an injection molding.

In some embodiments, the container holder comprises recesses or different thicknesses, which may serve for saving weight and material.

In some embodiments, the container holder is multi-piece. Thus, it may comprise e.g., a part manufactured by injection molding, e.g., an upper part, which is connected to, or lies on, a part component, e.g., a bottom part e.g., made of metal. Such a combination of material may include advantages which may encompass the high strength of metal and the cost-effective production of injection molding parts.

In some embodiments, the first container receptacle or the second container receptacle is a, preferably flat, support surface.

In some embodiments, the first container receptacle or the second container receptacle is only suitable and/or provided to position or touch a container, on one side thereof (preferably on the top, not also at the bottom).

In some embodiments, the container holder, the first container receptacle and/or the second container receptacle comprises an opening for inserting a container or a section thereof only on one side thereof (preferably on the top, not also at the bottom).

In some embodiments, the container holder comprises only one opening for inserting a container or a section thereof

In some embodiments, the container holder comprises only a tub-like section (e.g., a drip pan), in particular to collect liquid.

In some embodiments, the first and the second container receptacle comprise, for each container, support surfaces of different height, (the height is measured e.g., during use, e.g., from the bottom area or floor on which the dialysis apparatus carrying the container receptacle is placed).

In some embodiments, the container holder comprises no free-standing and/or upright pillars and/or no absorbent material.

In some embodiments, the container holder is not a disposable and/or not a modular article.

In some embodiments, the container holder comprises no cover, in particular no removable or welded cover, and/or no two-channel plug-in connectors.

In some embodiments, the container holder consists of or comprises thermoplastic. The latter may be polyamid-6 or polycaprolactam. The thermoplastic, in particular the polyamid-6, may be ball- and/or fiber-reinforced, e.g., with glass, carbon or other materials. The thermoplastic may be media-resistant.

The mounting device and the holding section may together enable a latching connection. They may enable a self-locking connection, which is in particular angle-dependent. They may comprise undercuts and/or may effect a mesh of one of these two components into the other.

The mounting device and the holding section may together comprise at least one tapering opening. They may comprise a mushroom-head lock.

In some embodiments, the container holder comprises a first and a second container. Optionally, it comprises no additional container besides these two containers.

In some embodiments, the first container comprises a liquid from the group of acidic solutions, in particular the acidic disinfectants. These are characterized in that they have a pH value less than 7.

The acidic solution may contain reducible ions, e.g., chloride-ions.

The liquid of the first container may comprise a single acid or a mixture of different acids, in particular from the group which consists of hydrochloric acid, acetic acid, citric acid, lactic acid, malic acid.

The liquid of the first container may be a mixture of citric acid, e.g., in the form of monohydrate, malic acid and lactic acid.

The liquid of the first container may be the disinfectants, as marketed by Fresenius Medical Care under the brand name Citrosteril.

The container containing this liquid may comprise a rectangular bottom area (or cross-sections).

In some embodiments, the second container comprises a liquid from the group of oxidizing solutions, in particular of the oxidizing disinfectants. These are characterized in that at least one component has an oxidizing effect on an acid-containing solution. Examples for acid-containing solutions are mentioned in the preceding paragraphs. The oxidizing solution may comprise one, several or all of the substances hypochlorite, peroxide, peracid.

The liquid of the second container may be an aqueous solution of sodium hypochlorite, potassium hydroxide and/or sodium hydroxide.

The liquid of the second container may be the disinfectant as marketed by Fresenius Medical Care under the brand name Sporotal.

The liquid of the first and/or second container is optionally no infusate, no dialysis liquid, no dialysate, no effluent and/or no substituate, or no precursor thereof.

The container containing this liquid may comprise an annular bottom area.

When removing the liquid from the containers, an uncontrolled leakage of the liquids out of the containers may occur, e.g., through sloshing or spilling out, adhesion of liquid to the removal device, overflowing. Hence, the two liquids may come into contact with each other. This may lead to dangers. The oxidizing agent may react with the acidic solution. Such a reaction may occur as follows, wherein toxicated chlorine gas is released:

2H₃0++OCl⁻+Cl⁻->3H₂0+Cl₂

In some embodiments, the container holder comprises no movable element, in particular relative to one of the support surfaces or container receptacles, in particular no movable holding section and/or no movable holding element.

In some embodiments, the container holder comprises no handle and/or no grip section protruding from the other parts of the container holder.

In some embodiments, the container holder comprises no device with which it supports itself directly (i.e. not via the dialysis apparatus) on the ground or floor, when it is connected to the dialysis apparatus. It comprises in particular no rollers.

In some embodiments, the container holder comprises no device with which it is in data/signal/fluid and/or energy transmission communication with the dialysis apparatus.

In some embodiments, the container holder and/or the container arranged thereon is not in a, for example, rigid fluid communication with the dialysis apparatus. In some embodiments, only one fluid transmission communication may be provided which is establishable or producible by or through or over a flexible tube may be provided.

In some embodiments, the holding section, which serves for connecting the container holder to the mounting device of the dialysis apparatus, is connected to or associated with,—preferably only—one of the container receptacles for positioning a respective container.

This container receptacle may be the first container receptacle. It may be the largest of all available container receptacles. It may be a container receptacle comprising no recess. It may be the container receptacle which is, during use, closest to the mounting device of the dialysis apparatus or arranged with no gap to the mounting device. As a result, a higher stability for arranging comparatively large containers may be achieved in each case. In addition, it may be ensured that liquid leaking from one of the containers may escape between dialysis apparatus and container holder, at least over sections of the container holder at which the latter has no holding sections, without wetting the dialysis apparatus.

In some embodiments, the holding section which serves for connecting the container holder to the mounting device of the dialysis apparatus, is arranged to be connected to a lower or middle section, but not to an upper section, of the mounting device.

In this way, in some embodiments, at least the upper section of the mounting device protrudes upwards above the holding section, above at least one container receptacle or above the entire container holder. As a result, it may be ensured that a liquid leaking from one of the containers wets only the mounting device but not the housing of the dialysis apparatus or components thereof (like connections, etc.). The mounting device, which is comparatively a small component, may be manufactured from material particularly suitable for contact with leaking liquid.

In some embodiments, the width of the container holder is between 30 cm and 50 cm. This enables arranging two containers next to each other on the container holder.

In some embodiments, the depth of the container holder is between 14 cm and 25 cm, preferably between 17 cm and 20 cm. This enables receiving the depth of a container in a comfortable manner.

In some embodiments, the depth of at least one of the container receptacles (from ground to the upper edge) is at least 8 cm, preferably 9 cm, 10 cm, 11 cm or more. This enables the container holder to keep e.g., a 1-liter container in the container receptacle in a tilt-proof manner.

In some embodiments, the container holder comprises at least one plane surface having a width of at least 15 cm, preferably at least 17 cm, and a depth of at least 15 cm, preferably 17 cm. This enables the container holder to comfortably position containers having a capacity of more than one liter.

Some or all embodiments may comprise one, several or all of the above-mentioned and/or the following advantages.

One advantage achievable by certain embodiments is that, liquids, like e.g., disinfectants, which spill or overflow from the container arranged on the container holder do not come into contact with each other. Chemical reactions between them may thus be advantageously prevented. The aforementioned partition wall, the drip pan and further features mentioned herein may contribute thereto.

A further advantage may be that due to the low arrangement of the retaining section at the dialysis apparatus, the stability of said arrangement is not impaired due to arranging or placing the containers. One notices how comfortable this may be when rolling the dialysis apparatus.

The provided partition walls may offer a lateral support to the containers placed on the container holder. These containers may hereby be prevented from falling, e.g., when rolling the dialysis apparatus.

Due to the movable holding, it may preferably be possible to arrange the container holder, in a space-saving manner, e.g., for its transport—for example at the dialysis apparatus—or to remove it therefrom.

The holding section for the movable holding of the container holder, in particular being a manually removable or releasable holding section, may enable an easy cleaning of the container holder and/or of the dialysis apparatus.

The movably fixed container holder may also comprise only one support surface. All of the explanations which relate to possible embodiments of the container holder and/or of the holding of the container holder at the dialysis apparatus, also apply to embodiments in which the container holder comprises only one support surface (e.g., only the first or only the second) and/or only one container receptacle (e.g., only the first or only the second).

The container holder comprises in some embodiments only one container receptacle (e.g., only the first or only the second), which however is designed with more than one support surface and/or designed for positioning or placing more than one container.

### BRIEF DESCRIPTION OF THE FIGURES

In the following, the present invention is exemplarily explained with regard to the accompanying drawing in which identical reference numerals refer to the same or similar elements. Wherein:

FIG. 1 shows a container holder according to the present disclosure in a first exemplary embodiment;

FIG. 2 shows the container holder of FIG. 1 according to the present disclosure in another perspective;

FIG. 3 shows a dialysis apparatus according to the present disclosure;

FIG. 4a, 4b show, in a sectional view, the container holder and the mounting device of the preceding figures in a use-mode (FIG. 4a) or during its connection with the dialysis apparatus or its release therefrom;

FIG. 5 shows an exemplary design of the container holder according to the present disclosure in a second embodiment; and

FIG. 6 shows a sectional view of the container holder of FIG. 5.

### DETAILED DESCRIPTION OF THE FIGURES

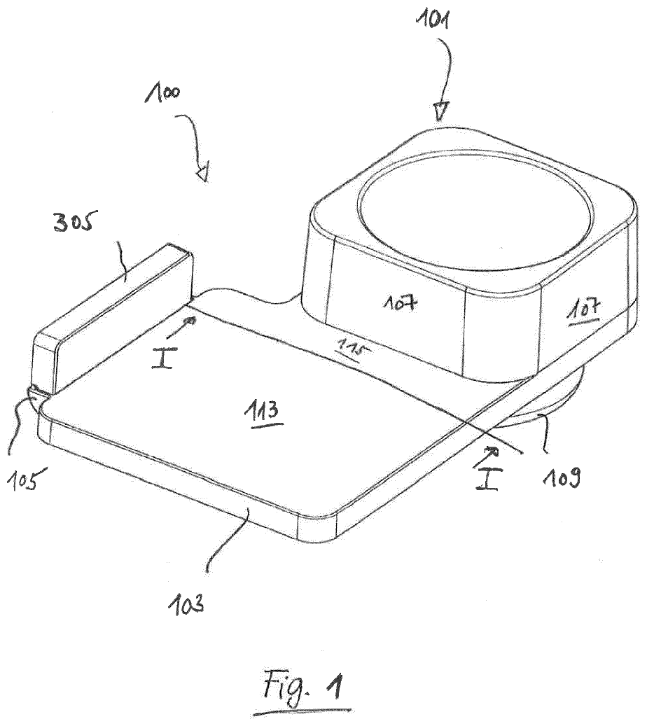

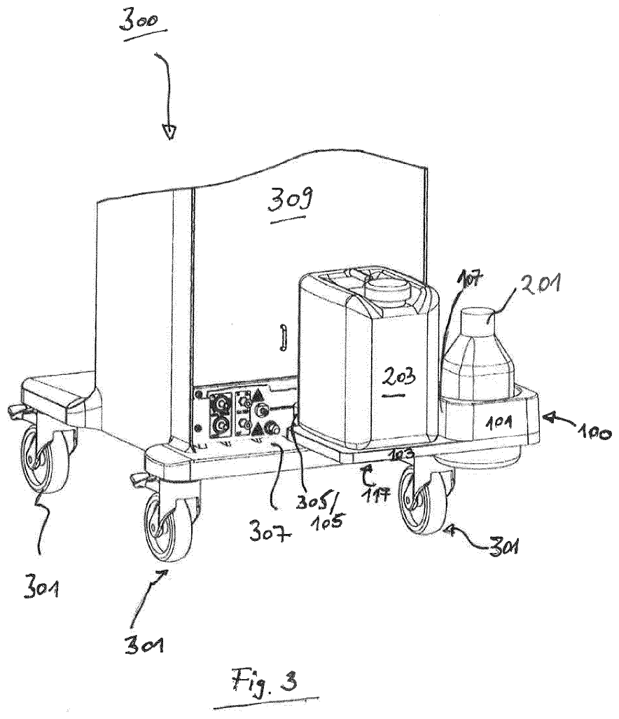

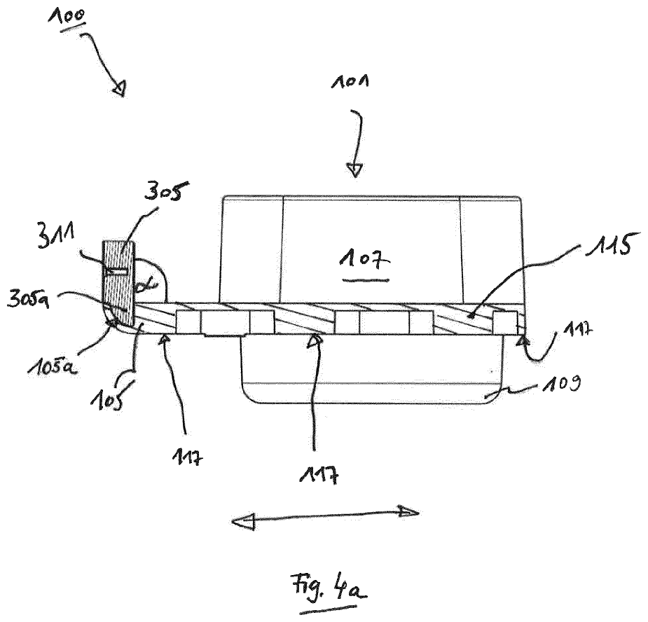

FIG. 1 shows a first embodiment of the container holder 100 according to the present disclosure. It comprises a first container receptacle 101 for positioning a first container (not shown in FIG. 1, see however the reference numeral 201 in FIG. 3) and a second container receptacle 103 for positioning a second container (not shown in FIG. 1, see however the reference numeral 203 in FIG. 3).

The container holder 100 further comprises a holding section 105 for holding the container holder 100 to the dialysis apparatus (not shown in FIG. 1, see however the reference numeral 300 in FIG. 3) in a movable, here releasable, manner. The holding section 105 is designed to be connected to a mounting device 305 of the dialysis apparatus 300. It serves the releasable connection of the container holder 100 to a retaining section of the dialysis apparatus 300.

The first container receptacle 101 is designed as an area surrounded by a partition wall 107 and open to the top, herein also referred to as an interior.

The partition wall 107 may in each embodiment be circumferential, i.e. closed. Alternatively, there may be a plurality of partition walls 107 provided, which are arranged apart from each other.

Partition walls 107 may in particular be provided between the first container receptacle 101 and the second container receptacle 103.

Partition walls 107 may extend in such way that liquids present on the bottom of one of the container receptacles 101, 103, are prevented from flowing or leaking into the other container receptacle, respectively.

Said area may, as optionally shown in FIG. 1, be limited to the bottom by a bottom section 109. It may also be closed to the bottom by the support surface such that liquids cannot leave this area downwards. The bottom section 109 may be designed as a drip pan.

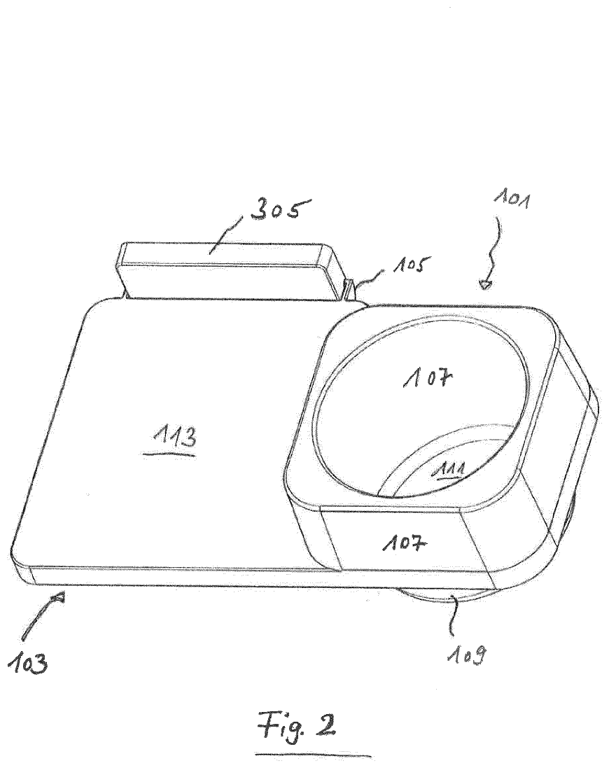

The bottom section 109 may comprise at its upper side a first support surface 111 (as seen in FIG. 2) for placing the first container 201. The first support surface 111 may be planar or curved. It may be free of elevations. It may comprise a through-opening with a closure device.

The second container receptacle 103 comprises an upper side which serves for placing the second container 203, not shown in FIG. 1. Therefore, the upper side is designated as a second support surface 113. It may be planar or curved. It may be free of elevations. It may comprise a through-opening with a closure device.

The first container receptacle 101 and the second container receptacle 103 are connected to each other by a connecting section 115. The connecting section 115 is placed between them. It may extend or pass seamlessly into the second container receptacle 103.

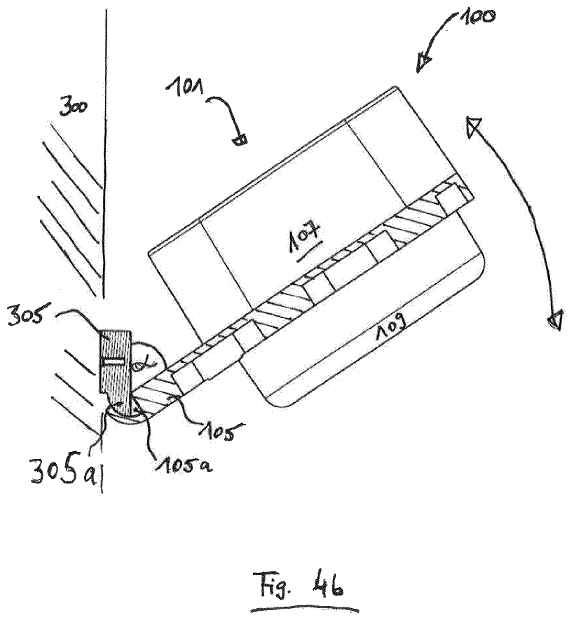

The releasable connection between the holding section 105 and the mounting device 305 is shown in detail in FIGS. 4a and 4b.

The holding section 105 and/or the mounting device 305 may each be one piece.

The holding section 105 and/or the mounting device 305 may each have a length of several centimeters. Due to their lengths, mechanical stress, which may result from the connection of the holding section 105 to mounting device 305, is distributed to a correspondingly large area.

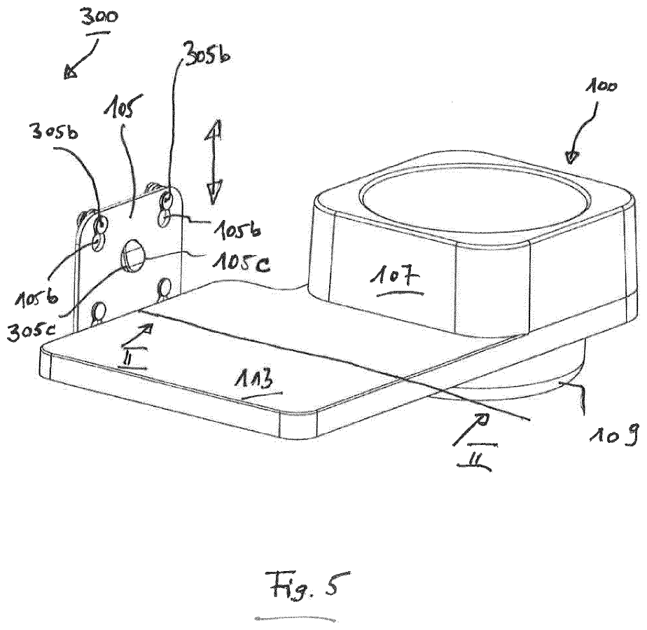

Unlike what is shown in FIG. 1 (and FIG. 5), a plurality of holding sections 105 and/or mounting devices 305 may be provided.

FIG. 2 shows the container holder 100 of FIG. 1 in another perspective.

The width of the container holder 100 extends in the right-left direction of FIG. 2. In FIG. 2, the depth extends (slightly distorted in perspective) from top to bottom. The container holder 100 may preferably be between 1 cm and 2 cm thick in the region of the first container receptacle 101.

FIG. 3 shows a dialysis apparatus 300 according to the present disclosure from the rear side a slight perspective from the top.

The dialysis apparatus 300 optionally comprises rollers 301, e.g., four. They serve for moving the dialysis apparatus 300 and may optionally comprise a blocking device.

The dialysis apparatus 300 comprises at its rear side a retaining section at which the mounting device 305 is arranged.

The retaining section is placed in the example of FIG. 3 in an area at the bottom side of the dialysis apparatus 300, e.g., slightly above the rollers 301.

The retaining section may optionally be so low that a bottom side 117 of the second container receptacle 103 touches a surface 307 of the dialysis apparatus 300 or its retaining section and/or supports itself thereon. The retaining section may advantageously serve for the secure and stable arrangement of the container holder 100 at the dialysis apparatus 300. In addition, such a support allows the use of correspondingly small-dimensioned and cost-effective components such as holding section 105 or mounting device 305.

The retaining section may serve for holding the rollers 301 to the other parts of the dialysis apparatus 300.

The retaining section may be a so-called supply rail, for example a section, at which one or more water inlets, one or more gas inlets, and/or one or more connections for a power supply are located. The supply rail may be an interface between the hydraulic system and external connections. It may have the function of introducing water and/or cleaning agents into the dialysis apparatus, conveying dialysis liquid to the patient (substitution) or to the dialyzer and discharging liquid from the dialyzer as well as steam from the machine.

As shown in FIG. 3, the container holder 100 may be fixed on the retaining section of the dialysis apparatus 300 such that only the second container receptacle 103 rests on the retaining section and/or is covered by or lies behind a rear wall 309 of the dialysis apparatus 300, while the first receptacle 101 protrudes laterally over the dialysis apparatus 300 or over its rear wall 309. With that, it may optionally be achieved that other inlets or accesses are free. These inlets may be part of the supply rail, by way of example. With that, optionally, it may also be achieved that one or several medium suction devices, which are arranged at the front side of the dialysis apparatus 300 and by use of which the content of the containers may be sucked out of the containers into the dialysis apparatus 300 through tube lines, do not require very long tubes. A lateral step may optionally further prevent a collision of the container with components at the rear wall of the dialysis apparatus 300. It may optionally enable the container to be visible from the front side of the dialysis apparatus 300 and semi-accessible and/or if necessary, prevent a collision of the partition wall 107 in the folded-up state with the rear wall of the dialysis apparatus 300. With that, more freedom may be given in determining a depth of the first container receptacle 101 extending in cross direction of the container holder 100. The depth extends in the direction of the double arrows shown in FIG. 4a. The depth of the first container receptacle 101 may, as shown in FIG. 3, be larger than the depth of the second container receptacle 103. It may also be however as large as or smaller than said depth of the second container receptacle 103.

FIG. 4a and FIG. 4b show the container holder 100 and the mounting device 305 of the preceding figures from the side and in a cut through the connecting section 115, the holding section 105 and the mounting device 305, along the line I-I of FIG. 1, i.e. with a view on the non-cut first container receptacle 101. Containers 201 or 203 are not shown.

FIG. 4a shows the holding section 105 and the mounting device 305 in a latched or engaged or completely connected state, as it appears when in use and, therefore, this state may optionally be understood to be the use-state.

In this, the end-side section 305a of the mounting device 305, which may be formed as a lug, engages in a recess 105a, which may be provided starting from an upper side of the holding section 105. This, or a differently designed, force-closure, and/or form-fit closure engagement of the mounting device 305 in the holding section 105 determines the container holder 100 at least in one direction which lies in the main extension plane of the container holder 100. In FIG. 4a, this direction is a right-left direction and designated by a double arrow.

As is seen in FIG. 4a, the end-side section 305a of the mounting device 305 may be recessed or offset by a step of further sections of the mounting device 305. It may have a rear side extending under a radium. It may have a straight front side.

The end-side section 305a of the mounting device 305 may be touched by or in contact with the respective surfaces of the recess 105a, at least at its end face and possibly at its side sections too.

The mounting device 305 may be prepared to be screwed to the retaining section of the dialysis apparatus 300. For this purpose, it may comprise one or several openings, e.g., thread openings 311. These openings may be through-openings or, as exemplarily shown in FIG. 4a, blind openings. In the example of FIG. 4a, the mounting device 305 is screwed from its rear side, i.e. from the side facing away from the container receptacle 101, 103. Other releasable or non-releasable connections between mounting device 305 and retaining section are likewise contemplated.

An angle α between holding section 105 and mounting device 305 is, during use, e.g., between 80° and 100°, approximately 90° or a right angle.

The mounting device 305 may be perpendicular to a base plane of the dialysis apparatus 300 and/or to a main extension plane of the container holder 100 during use thereof.

FIG. 4b shows the holding section 105 and the mounting device 305 in a non-latched or non-engaged or incompletely connected state. FIG. 4b does not show the container holder 100 during use. It rather shows a position of the latter when the container holder 100 is being connected to the dialysis apparatus 300 or released therefrom.

The angle α is less than 80°, here approximately 45°. It needs to be enlarged (up) to the angular dimension or extent shown in FIG. 4a in order to complete the connection of the container holder 100 to the dialysis apparatus 300.

As is seen in FIG. 4b, connecting the container holder 100 to the dialysis apparatus 300 or releasing it therefrom takes place by pivoting the container holder 100 in a direction shown by the double arrow in FIG. 4b.

The rotation center of the aforementioned pivotal movement lies, for simplicity, in the end-side section 305a, or near thereto.

FIG. 5 shows the container holder 100 according to the present disclosure in a second exemplary embodiment.

The container holder 100 of FIG. 5 corresponds, except for its holding section 105, to the container holder 100 of the first embodiment. The holding section 105 is, in the example of FIG. 5, not designed to be connected to the mounting device 305 by pivoting, unlike the example of FIG. 4a/FIG. 4b. Instead, the holding section 105 is connected to the mounting device 305 by moving it from top to bottom (relative to FIG. 5). In order to release it from the mounting device 305, it is moved (at least also) to the top (relative to FIG. 5). The double arrow of FIG. 5 indicates each movement.

As shown in FIG. 5, the holding section 105 comprises an opening 105b, in which a pin 305b of the mounting device 305 may engage. The opening 105b is designed as an opening tapering to the top. It may have the shape of a/an (inverted) key hole.

The pin 305b comprises a widening at its end side. It is exemplarily embodied having the shape of a mushroom head. Said widening fits through the non-tapered section of the opening 105b, but not through the tapered section thereof. In this way, a locking between pin 305b and opening 105b is achieved in a known manner.

The opening 105b may be designed as a through-opening as exemplarily shown in FIG. 5, but may also be a blind opening.

FIG. 5 shows four openings 105b. Some container holders according to the present disclosure have more or fewer such openings. The same applies for the number of pins 305b.

FIG. 5 further shows an optional opening 105c of the holding section 105 which opening is preferably designed as a through-opening. The opening 105c serves to receive a push pin, push bolt or a push button 305c, each of which is provided at the apparatus's side.

Unlike the openings 105b (relative to the pins 305b), the opening 105c is not designed to be movable relative to the push button 305c in order to separate the push button 305c from the opening 105c. In fact, the opening 105c should prevent a shifting of the holding section 105 relative to the mounting device 305 as long as the push button 305c extends into the opening 105c. Hence, the push button 305c stuck through the opening 105c prevents an unintentional release of the holding section 105 from the mounting device 305, for example due to a foot kick against the bottom side of the container holder 100, and prevents that the latter is unintentionally released from the dialysis apparatus 300.

In order to release the container holder 100 from the dialysis apparatus 300, the push button 305c has to be initially pushed, possibly against a resistance which may only be generated by a spring device, for example only so that it is pushed out of the opening 105c. Only when said push button 305c is not engaged with the opening 105c, the container holder 100 may be released from the pins 305b and thus from the dialysis apparatus 300 by shifting.

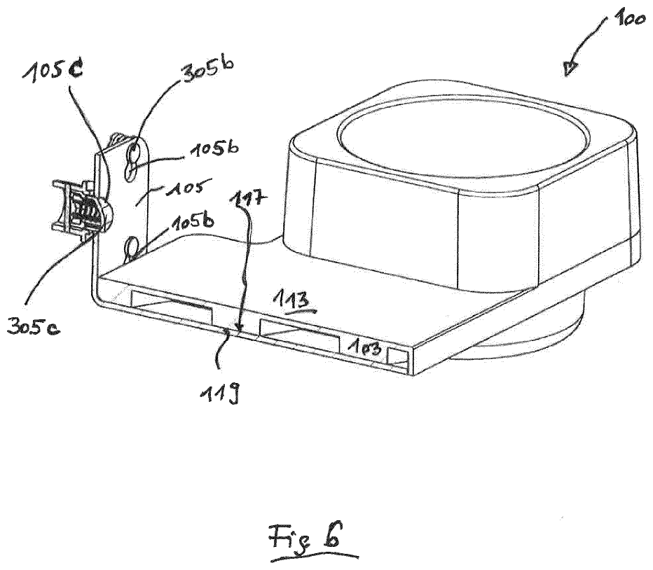

FIG. 6 shows the container holder 100 of FIG. 5 being cut along the line II-II of FIG. 5.

As can be seen, the holding section 105 continues in a bottom plate 119 on which the second container receptacle 103, which was manufactured separately from the bottom plate 119, is arranged or rests with its bottom side 117.

The person skilled in the art recognizes that it is irrelevant for the function intended whether the pins 305b and/or the push button 305c are arranged on the side of the dialysis apparatus 300 and the corresponding openings 105b and 105c, respectively, are arranged on the side of the container holder 100, or vice versa. The same applies for the components 105 and 305 of the embodiments shown in FIG. 1. They also may each be provided alternatively on the container holder 100 or on the dialysis apparatus 300.

As can be seen in the figures, the container holder may comprise sections with different depths being recognizable especially in a top view, see e.g., FIG. 2 which is very close to a top view.

### LIST OF REFERENCE NUMERALS

100 container holder
101 first container receptacle
103 second container receptacle
105 holding section
105a recess
105b opening
105c (optional) opening
107 partition wall
109 bottom section
111 first support surface
113 second support surface
115 connecting section
117 bottom side
119 bottom panel or plate
201 first container
203 second container
300 dialysis apparatus
301 rollers
305 mounting device
305a end-side section
305b pin
305c push button
307 surface
309 rear wall
311 thread opening
α angle between holding section and mounting device

1-13. (canceled)

14. A container holder for a dialysis apparatus, comprising:
a first container receptacle configured to position a first container;
a second container receptacle configured to position a second container; and
a holding section configured to movably connect the container holder to the dialysis apparatus,
wherein at least one partition wall is arranged between the first container receptacle and the second container receptacle.

15. The container holder according to claim 14, wherein the first container receptacle comprises:
a drip pan configured to collect liquid leaking or originating from the first container while the first container is in the first container receptacle.

16. The container holder according to claim 15, wherein the second container receptacle comprises:
a drip pan configured to collect liquid leaking or originating from the second container while the second container is in the second container receptacle.

17. The container holder according to claim 14, wherein at least a section of a first support surface of the first container receptacle is arranged lower than at least a section of a second support surface of the second container receptacle, wherein the section of the first support surface is configured to position the first container, and wherein the second support surface is configured to position the second container.

18. The container holder according to claim 14, wherein at least a section of a first support surface of the first container receptacle and at least a section of a second support surface of the second container receptacle are arranged lower than a connecting section between the first container receptacle and the second container receptacle.

19. The container holder according to claim 14, wherein at least a section of a first support surface of the first container receptacle or at least a section of a second support surface of the second container receptacle is arranged lower than a connecting section between the first container receptacle and the second container receptacle.

20. The container holder according to claim 14, wherein an upper edge of the first container receptacle is arranged higher than a second support surface of the second container receptacle, and wherein the second support surface is configured to position the second container.

21. The container holder according to claim 14, wherein the holding section comprises a folding device, a shifting device, and a releasable mounting device.

22. The container holder according to claim 14, wherein the holding section comprises: a folding device, a shifting device, or a releasable mounting device.

23. The container holder according to claim 14, wherein the holding section comprises at least one opening configured to receive at least one of pins, push buttons, and securing pins.

24. The container holder according to claim 23, wherein the at least one opening comprises at least one round opening and at least one keyhole-formed or tapered opening.

25. The container holder according to claim 23, wherein the at least one opening comprises at least one round opening or at least one keyhole-formed or tapered opening.

26. The container holder according to claim 14, wherein the first container receptacle and the second container receptacle each comprise an opening or a free interior with an annular cross-section.

27. The container holder according to claim 14, wherein the first container receptacle or the second container receptacle comprises an opening or a free interior with an annular cross-section.

28. The container holder according to claim 14, wherein at least a section of a first support surface of the first container receptacle or at least a section of a second support surface of the second container receptacle is arranged lower than a connecting section between the first container receptacle or the second container receptacle.

29. The container holder according to claim 14, wherein at least a section of a first support surface of the first container receptacle and at least a section of a second support surface of the second container receptacle are arranged lower than a connecting section between the first container receptacle or the second container receptacle.

30. A dialysis apparatus comprising:
a container holder comprising:
a first container receptacle configured to position a first container;
a second container receptacle configured to position a second container; and
a holding section,
wherein at least one partition wall is arranged between the first container receptacle and the second container receptacle;
a retaining section; and
a mounting device attached to the retaining section,
wherein the holding section of container holder is connected to the retaining section by the mounting device at a movable, shiftable, or releasable connection.

31. A dialysis apparatus according to claim 30, wherein the mounting device comprises at least one pin and securing pin.

32. The dialysis apparatus according to claim 30, wherein the mounting device comprises at least one push button.

33. The dialysis apparatus according to claim 30, wherein the moveable, shiftable, or releasable connection is foldable.
